(19) United States
(12) Patent Application Publication
(10) Pub. No.: US 2005/0154430 A1
(43) Pub. Date: Jul. 14, 2005

(54) Transpulmonary systemic cooling using liquid mists

(75) Inventors: Denise R. Barbut, New York, NY (US); Jean G. Riess, Falicon (FR); N. Simon Faithfull, Bottesford (GB)

Correspondence Address: O'MELVENY & MEYERS, 114 PACIFICA, SUITE 100, IRVINE, CA, 92618, US

(73) Assignee: BENECOOL, Inc., New York, NY (US)

(21) Appl. No.: 10/792,365

(22) Filed: Mar. 2, 2004

Related U.S. Application Data
(60) Provisional application No. 60/535,230, filed on Jan. 9, 2004.

(51) Int. Cl. A61F 7/00

(52) U.S. Cl. 607/96

(57) ABSTRACT

A method for transpulmonary cooling by providing a liquid having a boiling point of 38-300° C., more preferably 38-250° C., more preferably 38-200° C., more preferably 38-150° C., more preferably 38-80° C. The liquid is nebulized to form a mist. The mist is optionally cooled below room temperature and delivered to the airway of a patient so that the patient inhales the mist. The mist causes systemic cooling by evaporative heat loss when inhaled at room temperature and additionally by direct heat transfer when inhaled below room temperature. Compositions and medical devices for transpulmonary cooling are also provided.

### CROSS-REFERENCE TO RELATED APPLICATION

This application claims the benefit pursuant to 35 U.S.C. § 119(e) of U.S. Provisional Patent Application Ser. No. 60/535,230, filed Jan. 9, 2004, which is incorporated herein by reference in its entirety.

### FIELD OF THE INVENTION

The invention relates to transpulmonary systemic cooling, and more particularly to transpulmonary systemic cooling using liquids or liquid mists with boiling points above body temperature.

### BACKGROUND

Patients experiencing cerebral ischemia often suffer from disabilities ranging from transient neurological deficit to irreversible damage (stroke) or death. Cerebral ischemia, i.e., reduction or cessation of blood flow to the central nervous system, can be characterized as either global or focal. Global cerebral ischemia refers to reduction of blood flow within the cerebral vasculature resulting from systemic circulatory failure caused by, e.g., shock, cardiac failure, or cardiac arrest. Within minutes of circulatory failure, tissues become ischemic, particularly in the heart and brain.

The most common form of shock is cardiogenic shock, which results from severe depression of cardiac performance. The most frequent cause of cardiogenic shock is myocardial infarction with loss of substantial muscle mass. Pump failure can also result from acute myocarditis or from depression of myocardial contractility following cardiac arrest or prolonged cardiopulmonary bypass. Mechanical abnormalities, such as severe valvular stenosis, massive aortic or mitral regurgitation, acutely acquired ventricular septal defects, can also cause cardiogenic shock by reducing cardiac output. Additional causes of cardiogenic shock include cardiac arrhythmia, such as ventricular fibrillation.

With sudden cessation of blood flow to the brain, complete loss of consciousness is a sine qua non in cardiac arrest. Cardiac arrest often progresses to death within minutes if active interventions, e.g., cardiopulmonary resuscitation (CPR), defibrillation, use of inotropic agents and vasoconstrictors such as dopamine, dobutamine, or epinephrine, are not undertaken promptly. The most common cause of death during hospitalization after resuscitated cardiac arrests is related to the severity of ischemic injury to the central nervous system, e.g., anoxic encephalopathy. The ability to resuscitate patients of cardiac arrest is related to the time from onset to institution of resuscitative efforts, the mechanism, and the clinical status of the patient prior to the arrest.

Focal cerebral ischemia refers to cessation or reduction of blood flow within the cerebral vasculature resulting in stroke, a syndrome characterized by the acute onset of a neurological deficit that persists for at least 24 hours, reflecting focal involvement of the central nervous system. Approximately 80% of the stroke population is hemispheric ischemic strokes, caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. Hemorrhagic stroke accounts for the remaining 20% of the annual stroke population. Hemorrhagic stroke often occurs due to rupture of an aneurysm or arteriovenous malformation bleeding into the brain tissue, resulting in cerebral infarction. Other causes of focal cerebral ischemia include vasospasm due to subarachnoid hemorrhage from head trauma or iatrogenic intervention.

Current treatment for acute stroke and head injury is mainly supportive. A thrombolytic agent, e.g., tissue plasminogen activator (t-PA), can be administered to non-hemorrhagic stroke patients. Treatment with systemic t-PA is associated with increased risk of intracerebral hemorrhage and other hemorrhagic complications. Aside from the administration of thrombolytic agents and heparin, there are no therapeutic options currently on the market for patients suffering from occlusion focal cerebral ischemia. Vasospasm may be partially responsive to vasodilating agents. The newly developing field of neurovascular surgery, which involves placing minimally invasive devices within the carotid arteries to physically remove the offending lesion, may provide a therapeutic option for these patients in the future, although this kind of manipulation may lead to vasospasm itself.

Cooling has also been shown to be beneficial in patients undergoing neurosurgical procedures for ruptured aneurysms, and in patients undergoing coronary bypass surgery. In such cases, the protection provided is for the brain. Cooling may also be beneficial for myocardial protection during myocardial ischemia.

In both stroke and cardiogenic shock, patients develop neurological deficits due to reduction in cerebral blood flow. Treatments should include measures to maintain viability of neural tissue, thereby increasing the length of time available for interventional treatment and minimizing brain damage while waiting for resolution of the ischemia. New devices and methods are thus needed to minimize neurologic deficits in treating patients with either stroke or cardiogenic shock caused by reduced cerebral perfusion.

### SUMMARY OF THE INVENTION

The compositions, methods, and devices described herein have significant and unexpected advantages over earlier attempts for transpulmonary systemic cooling. Earlier attempts suffer from at least four disadvantages. First, the earlier attempts have a tendency to cause air trapping in the lungs, which is harmful. Second, for compounds with low boiling points, explosive evaporation causing barotrauma has proven to be problematic. Third, delivery is problematic with low boiling compounds because they vaporize before reaching the lower airways. Finally, hypoxia has been noted to be a problem with earlier attempts. Hypoxia occurs when a vaporized gas other than oxygen is present in the lungs and dilutes other gasses present in the lungs. When hypoxia occurs, it becomes necessary to increase the inspired oxygen fraction.

The invention relates to methods, devices, and compositions for transpulmonary cooling. The compositions of the invention include liquids having a boiling point of 38-300° C., more preferably a boiling point of 38-200° C., more preferably a boiling point of 38-150° C., more preferably a boiling point of 38-125° C., more preferably a boiling point of 38-110° C. Compounds having suitable characteristics for use herein include hydrocarbons, fluorocarbons, perfluorocarbons, and perfluorohydrocarbons. As used in this specification, the terms “fluorocarbon,” “perfluorocarbon,” and “perfluorohydrocarbon” are synonymous. In addition to containing carbon and fluorine, these compounds may also contain other atoms. In one embodiment, the compounds could contain a heteroatom, such as nitrogen, oxygen, or sulfur, or a halogen, such as bromine or chlorine. These compounds may be linear, branched, or cyclic, saturated or unsaturated, or any combination thereof.

In another embodiment, the compounds are highly fluorinated compounds, which are compounds containing at least three fluorine atoms. These highly fluorinated compounds may also contain other atoms besides carbon and fluorine. These other atoms include, but are not limited to, hydrogen; heteroatoms such as oxygen, nitrogen, and sulfur; and halogens such as bromine or chlorine. In one embodiment, the number of the atoms that are not carbon or fluorine comprise a minority of the total number of atoms in the compound. These highly fluorinated compounds may be linear, branched, or cyclic, saturated or unsaturated, or any combination thereof. Examples of these compounds include, but are not limited to, C₄F₉Br (b.p. 43° C.), CF₃CF(CF₃)CF═CF₂ (b.p. 51° C.), CF₃CF(CF₃)CH═CH₂,

In another embodiment, the compounds are hydrofluorocarbons, which are compounds where the number of hydrogen atoms exceeds the number of fluorine atoms. These hydrofluorocarbons may also contain other atoms besides hydrogen, carbon, and fluorine. These other atoms include, but are not limited to, heteroatoms such as oxygen, nitrogen, and sulfur and halogens such as chlorine and bromine. For example, hydrofluorcarbons include, but are not limited to, hydrochlorofluorocarbons, more specifically, hydrochlorofluoralkanes. In one embodiment, the number of the atoms other than carbon and fluorine comprise a minority of the total number of atoms in the compound. These hydrofluorocarbons may be linear, branched, or cyclic, saturated or unsaturated, or any combination thereof.

A mixture of two or more highly fluorinated compounds, hydrofluorocarbons, light fluorocarbons, hydrocarbons, fluorocarbons, perfluorocarbons, perfluorohydrocarbons, or any of the above-mentioned compounds may also be used. The mixture may contain any of the previously mentioned compounds in different phases (e.g., one gas, one liquid). The mixture has a boiling point above 37° C., even though any individual component of the mixture may have a boiling point below 37° C.

Light fluorocarbons are fluorocarbons that have a boiling point below 37° C. These light fluorocarbons may also contain other atoms besides carbon, and fluorine. These other atoms include, but are not limited to, hydrogen; heteroatoms such as oxygen, nitrogen, and sulfur; and halogens such as chlorine and bromine. For example, light fluorocarbons include, but are not limited to perfluorobutane and perfluoropentane. In one embodiment, the number of the atoms other than carbon and fluorine comprise a minority of the total number of atoms in the compound. These light fluorocarbons may be linear, branched, or cyclic, saturated or unsaturated, or any combination thereof.

In certain methods, a liquid having a boiling point of 38-300° C., more preferably having a boiling point of 38-200° C., more preferably having a boiling point of 38-150° C., is selected. The liquid is nebulized to form a mist. The droplets preferably range in size from 0.1-100 microns, more preferably 1-5 microns, more preferably 2-4 microns. The mist is optionally cooled below body temperature and delivered to the airway of a patient so that the patient inhales the mist. Inhalation of the mist causes systemic cooling by heat transfer from the lungs to the cooler mist and/or by evaporative heat loss as the mist evaporates. The administration of the liquid is continued until the systemic temperature is reduced to 35° C. or below, more preferably to 34° C. or below, more preferably to 33° C. or below. The rate of cooling can be adjusted by varying the temperature of the inhalate, the concentration of the responsible compound or compound mixture, the rate of delivery, the particle size, and the percentage of each compound in the mixture.

In another embodiment, the liquid is administered directly to the patient. In some circumstances, it may not be necessary to nebulize the liquid. For example, in patients already supplied with an endotracheal tube, pure liquid may be introduced with or without the techniques of partial or total liquid ventilation.

Medical devices are also provided for transpulmonary cooling. The devices include an inhaler device and a nebulized liquid in the form of a mist, the liquid having a boiling point of 38-300° C., more preferably having a boiling point of 38-200° C., more preferably having a boiling point of 38-150° C. Any of the biocompatible liquids having boiling points within the ranges described herein are suitable for use with the medical devices described herein. The liquid mist may be cooled to below body temperature before delivery. The mist droplets may range in size from 0.1-100 microns, more preferably 1-5 microns, more preferably 2-4 microns.

### DETAILED DESCRIPTION

The compositions of the invention include liquids having a boiling point above 37° C. and less than or equal to 300° C., more preferably 38-300° C., more preferably 38-200° C., more preferably 38-150° C., more preferably 38-100° C., more preferably 38-80° C., more preferably 40-150° C., more preferably 40-100° C., more preferably 40-75° C., more preferably 45-150° C., more preferably 45-100° C., more preferably 45-75° C., more preferably 50-150° C., more preferably 50-100° C., more preferably 50-75° C., more preferably 50-70° C. Compounds having suitable characteristics for use herein include, but are not limited to, highly fluorinated compounds, hydrofluorocarbons, hydrocarbons, fluorocarbons, perfluorocarbons, and perfluorohydrocarbons. Suitable biocompatible liquids include perfluorohexane (b.p. 57° C.), perfluorocyclohexane (b.p. 53° C.), and perfluoroethers selected from the group comprising of (C₃F₇)₂O (b.p. 56° C.), CF₃(OCF₂)₃OCF₃ (b.p. 59° C.), C₃F₇—O—C₃F₇ (b.p. 57° C.), (CF₃OCF₂CF₂)₂O (perfluorodiglyme, b.p. 66° C.), CF₃(OCF₂)₃OCF₃ (b.p. 59° C.), and the hydrofluoroethers C₄F₉OCH₃ (b.p. 60° C.), C₄F₉OC₂H₅(b.p. 76° C.), perfluoro(n-butyl)tetrahydrofurane C₈F₁₆O (b.p. 97-107° C.), perfluoro-2-(n-butyl)tetrahyrofurane, perfluoro-3-(n-butyl)tetrahyrofurane, and others. Further valuable highly fluorinated components include mixed fluorocarbon-hydrocarbon diblock compounds such as, for example, CₙF₂ₙ₊₁CₘH₂ₘ₊₁ or CₙF₂ₙ₊₁OCₘH₂ₘ₊₁.

Moreover, a mixture of two or more fluorocarbons or highly fluorinated compounds, or a mixture of two or more fluorocarbons and hydrofluorocarbons, may also be used, including mixtures of any of the above-identified compounds. The mixture may further include compounds with boiling points below 37° C., provided the mixture itself has a boiling point that is above 37° C. For example, a mixture of perfluorohexane (PFH, b.p. 57° C.) and perfluoropentane (PFP, b.p. 29° C.) having a boiling point above 37° C. has suitable properties, and is within the scope of the present teaching. The proportions of any mixture of compounds may be varied during the procedure to achieve desired boiling point and vapor pressure characteristics. Moreover, the procedure may be commenced with a higher proportion of PFP (b.p. closer to 29° C.), then to maintain the cooling, the composition can be enriched with a greater proportion of PFH (b.p. closer to 50° C.). The proportions may be varied during the procedure by administering different proportions at different time points. Alternatively, or in addition, the composition may be varied automatically as a result of preferential evaporation of the more volatile components in the body.

In certain methods, the liquid may be cooled to below body temperature before delivery. The liquid or liquid mixture may be cooled to 35° C. or below, 30° C. or below, 25° C. or below, 20° C. or below, 15° C. or below, or 10° C. or below. This pre-cooling will promote a more rapid transpulmonary systemic cooling and reduce the total amount of fluorocarbon required to achieve a set amount of cooling.

In a first method, a liquid having a boiling point of 38-300° C., more preferably having a boiling point of 38-200° C., more preferably having a boiling point of 38-150° C., is selected. The liquid is nebulized to form a mist. The droplets preferably range in size from 0.1-100 microns, more preferably 0.1-20 microns, more preferably from 1-5 microns, more preferably from 2-4 microns. The mist is delivered to the airway of a patient so that the patient inhales the mist. Inhalation of the mist causes systemic cooling by heat transfer from the cooler mist and/or by evaporative heat loss. The volume of liquid administered typically ranges from 1 to 6 liters or more. In some cases, up to 10 and even 20 L may be administered. In other cases, 3 to 4 liters may be administered. In some cases, less than 1 liter of liquid may be administered, for example, 0.75 liters, more preferably 0.5 liters, more preferably 0.1 liters. This is especially the case if the fluorinated compound is not deposited into the lungs. Induction of cooling is rapid, occurring within 1 minute, 2 minutes, 4 minutes, 8 minutes, or over a longer time period such as under 30 minutes, under 60 minutes, or over 60 minutes ,depending on the composition, volume, and temperature of the mist administered. The administration of the liquid is continued until the systemic temperature is reduced to 35° C. or below, or more preferably to 34° C. or below. Moreover, the cooling can be maintained for a prolonged period, up to 4 hours or more, 8 hours or more, 12 hours or more, 16 hours or more, 24 hours or more, 36 hours or more, or 48 hours or more.

Medical devices are also provided for transpulmonary cooling. The devices include an inhaler device and a nebulized liquid in the form of a mist the liquid having a boiling point of 38-300° C., more preferably having a boiling point of 38-200° C., more preferably having a boiling point of 38-150° C. Any of the biocompatible liquids having boiling points within the ranges described herein are suitable for use with the medical devices described herein. The liquid mist may be cooled to below body temperature before delivery. In certain cases, the liquid mist is cooled to 35° C. or below, 30° C. or below, 25° C. or below, 20° C. or below, 15° C. or below, or 10° C. or below. The mist droplets may range in size from 0.1 to 100 microns, more preferably from 0.1-20 microns, more preferably from 1-5 microns, more preferably from 2-4 microns.

The mist may be delivered in a gaseous mixture containing oxygen, for example, 20% oxygen or more, as in inspired air. Alternatively, the mist may be delivered in a gaseous mixture containing increased fractions of oxygen, for example, more than 20% oxygen or more. The remaining inspired gas can include one or more gaseous fluorinated compound (any of those described herein, such as light fluorocarbons, hydrofluorocarbons or hydrochlorofluorocarbons) rather than nitrogen to increase the cooling capacity of the gaseous mixture, thus further reducing the amount of liquid fluorocarbon required. Other possible components of the gaseous mixture include, but are not limited to, nitrogen, CO₂, as present in carbogen, helium, etc. The fluorinated gas might also be SF₆, a substance approved for many other indications in humans.

In another embodiment, the fluorocarbons may be recovered from the expired gas. In some cases, the recovered fluorocarbons may be readministered to the patient. By recirculation, the total volume of fluorocarbon necessary to achieve systemic cooling can be vastly reduced.

Although the foregoing invention has, for the purposes of clarity and understanding, been described in some detail by way of illustration and example, it will be obvious that certain changes and modifications may be practiced which will still fall within the scope of the appended claims. It will also be understood that any feature or features from any one embodiment, or any reference cited herein, may be used with any combination of features from any other embodiment.

1. A method for transpulmonary cooling, comprising the steps of:
providing a liquid having a boiling point of 38-300° C.;
nebulizing the liquid to form a mist; and
delivering the mist to the airway of a patient so that the patient inhales the mist to cause systemic cooling.

2. The method of claim 1, wherein the liquid has a boiling point of 38-200° C.

3. The method of claim 1, wherein the liquid has a boiling point of 38-150° C.

4. The method of claim 1, wherein the liquid or liquid mist is cooled to below body temperature before delivery.

5. The method of claim 1, wherein the liquid or liquid mist is cooled to 10° C. or less before delivery.

6. The method of claim 1, wherein the liquid comprises at least one highly fluorinated compound.

7. The method of claim 6, wherein the at least one highly fluorinated compound comprises a linear compound.

8. The method of claim 6, wherein the at least one highly fluorinated compound comprises a branched compound.

9. The method of claim 6, wherein the at least one highly fluorinated compound comprises a cyclic compound.

10. The method of claim 6, wherein the at least one highly fluorinated compound comprises a saturated compound.

11. The method of claim 6, wherein the at least one highly fluorinated compound comprises an unsaturated compound.

12. The method of claim 6, wherein the at least one highly fluorinated compound comprises at least one heteroatom.

13. The method of claim 6, wherein the at least one highly fluorinated compound comprises at least one hydrogen.

14. The method of claim 6, wherein the highly fluorinated compound comprises at least one halogen.

15. The method of claim 1, wherein the liquid is a fluorocarbon.

16. The method of claim 6, wherein the highly fluorinated compound is a perfluoroalkane of the formula CₙF₂ₙ₊₂.

17-29. (canceled)

30. The method of claim 1, wherein the liquid mist further comprises at least one additional fluorinated component.

31. The method of claim 30, wherein the at least one additional fluorinated component boils below 37° C.

32-63. (canceled)

64. A composition for transpulmonary cooling, comprising:
a nebulized liquid in the form of a mist, the liquid having a boiling point of 38-300° C.

65-111. (canceled)

112. A medical device for transpulmonary cooling, comprising:
an inhaler device; and
a nebulized liquid in the form of a mist the liquid having a boiling point of 38-300° C.

113-149. (canceled)

150. A method for transpulmonary cooling, comprising the steps of:
providing a liquid fluorocarbon;
nebulizing the liquid to form a mist;
delivering the mist to the airway of a patient so that the patient inhales the mist to cause systemic cooling;
recovering the fluorocarbon from an expired gas; and
recirculating the recovered fluorocarbon to the patient.

151. (canceled)
